(19) United States
(12) Patent Application Publication
(10) Pub. No.: US 2021/0098144 A1
(43) Pub. Date: Apr. 1, 2021

(54) METHOD FOR MANUFACTURING LEAD-FREE RADIATION SHIELDING SHEET AND LEAD-FREE RADIATION SHIELDING SHEET

(71) Applicant: DR-VU CO., LTD., Seoul (KR)

(72) Inventor: Eun Jung PARK, Seoul (KR)

(73) Assignee: DR-VU CO., LTD., Seoul (KR)

(21) Appl. No.: 16/970,634

(22) PCT Filed: Jan. 8, 2020
(86) PCT No.: PCT/KR2020/000334

§ 371 Date: Aug. 18, 2020

(30) Foreign Application Priority Data
Jan. 16, 2019 (KR) 10-2019-0005743

(51) Int. Cl. G21F 1/12

(52) U.S. Cl. CPC G21F 1/12; G21F 1/08

(57) ABSTRACT

The present invention discloses a method for manufacturing a lead-free radiation shielding sheet. The method for manufacturing a lead-free radiation shielding sheet according to the present invention comprises a film laminating step of forming a multi-layered radiation shielding film on one side of a base material by repeating a process of applying to laminate, drying, and integrating a radiation shielding material containing a radiation shielding powder and a binder for forming a film to be mixed with each other on one side of the base material for forming a radiation shielding sheet. According to the present invention, since heavy lead which is harmful to the human body and the environment is not used, side effects such as disease or environmental pollution caused by lead do not occur, the light weight of the radiation shielding sheet is enabled, and protective clothing with excellent wearing sensation can be manufactured, and since flexibility can be improved compared to lead rubber sheets, handling and storage are convenient. In addition, the lead-free radiation shielding sheet manufactured by the present invention can be applied as clothes of various designs and radiation protection means for various uses due to flexibility and ease of operation.

### TECHNICAL FIELD

The present invention relates to a method for manufacturing a lead-free radiation shielding sheet and a lead-free radiation shielding sheet manufactured by the same, and more particularly, to a method for manufacturing a lead-free radiation shielding sheet that does not contain a lead component and can be compactified in thickness, and a lead-free radiation shielding sheet having a multi-layered structure manufactured by the same.

### BACKGROUND ART

In places where radiation is generated or present, for example, X-ray imaging and radiation therapy rooms in hospitals, radiation zones of nuclear power plants, radiation transmission laboratories, sites of handling x-ray clearance inspection equipment, etc., the risk and anxiety of the human body for radiation exposure are amplified, and interest in radiation exposure is increasing in recent years.

In general, when radiation such as X-rays and gamma rays is exposed to humans, it is known that the risk of causing various serious diseases and disorders such as carcinogenesis, genetic disorders, and cataracts increases.

Accordingly, the International Commission on Radiological Protection was established in 1934 to limit the use of radiation (0.2 R/day), and in 1977, the advice (ICRP-26) of the International Commission on Radiological Protection was adopted. Subsequently, guidelines have been published to reduce the exposure of patients, workers and caregivers to X-ray diagnosis, treatment and nuclear medicine, and act on the regulation of radiation use has been established in each country.

As described above, since the radiation exposure is very harmful to the human body, the radiation exposure should be made as limited as possible, but particular attention should be paid to those who directly or indirectly treat radiation, such as radiologists, physicians, and nurses in hospitals, and nuclear power plant-related persons, because they may be exposed to radiation continuously due to the nature of their work.

In addition, even in patients receiving radiographic imaging or radiation treatment due to disease, radiation exposure more than necessary should be minimized or prevented, and it is preferable that sites other than a site to be examined or treated, that is, a target site, or human tissues such as organs vulnerable to radiation, etc. are properly protected from radiation.

Currently, persons who work in places exposed frequently to radiation, such as repair or inspection of nuclear power plants, wear radiation shielding clothing (radiation protective clothing) to protect the human body from the risk of exposure, and radiation protection clothing for radiologists and patients is provided even in hospitals.

As a method for shielding radiation exposure, it is common to wear protective clothing such as a gown (radiation protective gown) to which a sheet (lead rubber) formed by dispersing and extruding a lead component in rubber (rubber) is applied.

The lead rubber is also called rubber lead, as a rubber containing a large amount of lead component, and is usually manufactured in the form of a sheet to be applied to radiation protection products. The radiation protection products applied with the lead rubber includes lead-rubber apron, lead-rubber gloves, radiographic imaging clothes (radiation gowns), and other radiation work clothes.

Lead rubber, which is commonly used for radiation protection (shielding), is effective for shielding radiation, but it is very heavy, uncomfortable, and provides a firm wearing sensation. More specifically, since the radiation shielding sheet made of lead rubber has poor flexibility, is easily torn by bending, does not have sufficient friction resistance, that is, abrasion resistance, and has a heavy and hard texture (hardness), protective clothing applied with a radiation shielding sheet made of lead rubber is difficult to wear and movement is very uncomfortable when wearing the protective clothing.

In particular, the radiation used in hospitals has a relatively low dose compared to radiation generated in nuclear power plants, a low risk of direct radiation exposure, and a high risk of indirect exposure by radiation diffraction, but hospital officials need to wear radiation gowns applied with a heavy lead rubber sheet and take the inefficiencies of doing works.

In the case of a shielding sheet using lead as a main component, there is a problem in a risk of lead poisoning and environmental pollution, and lead poisoning has symptoms, such as speech disorder, headache, abdominal pain, anemia, and exercise paralysis. Lead may damage the nervous system to lose the brain's reaction and even lower its intelligence.

On the other hand, for lighter radiation shielding clothing, in U.S. Pat. No. 3,194,239, there is disclosed a method for manufacturing a radiation absorbing fiber using an alloy wire for absorbing radiation, but there is a problem of poor flexibility and radiation shielding property.

### DISCLOSURE

### Technical Problem

An object of the present invention is to provide a method for manufacturing a lead-free radiation shielding sheet capable of enabling proper radiation protection without using lead that is harmful to the human body and a radiation shielding sheet manufactured by the same.

A specific object of the present invention is to provide a method for manufacturing a lead-free radiation shielding sheet having a multi-layered structure which is thin and flexible and excellent in radiation shielding performance to the same thickness by using a radiation shielding material containing a radiation shielding power and a binder resin and a radiation shielding sheet manufactured by the same.

### Technical Solution

An aspect of the present invention provides a method for manufacturing a lead-free radiation shielding sheet comprising: a film laminating step of forming a multi-layered radiation shielding film on one side of a base material by repeating a process of sequentially applying to laminate, drying, and integrating a radiation shielding material containing a radiation shielding powder and a binder for forming a film to be mixed with each other on one side of the base material for forming a radiation shielding sheet.

The film laminating step may comprise a shielding material applying step of sequentially applying and drying the radiation shielding material on one side of the base material a plurality of times so that the radiation shielding film has a multi-layered structure of at least three layers.

In the shielding material applying step, an N-layered radiation shielding film may be formed by sequentially applying and drying the radiation shielding material on one side of the base material N (3≤N≤10) times.

The film laminating step may comprise a shielding material applying step of repeating a process of sequentially applying at a thickness of 0.05 mm to 0.50 mm and drying the radiation shielding material on one side of the base material a plurality of times.

The shielding material applying step may also comprise an inner film forming step of performing a process of sequentially applying at a thickness of 0.1 mm to 0.30 mm and drying the radiation shielding material on one side of the base material at least twice. The film forming step is a step of forming an inner shielding film performed before forming a layer (surface shielding film) of forming the last radiation shielding film, that is, the surface of the radiation shielding film to be formed last in the film laminating step.

The shielding material applying step may comprise a first film forming step of forming a first shielding film by applying at a thickness of 0.1 mm to 0.3 mm and drying the radiation shielding material on one side of the base material; a second film forming step of forming a second shielding film by applying at a thickness of 0.1 mm to 0.3 mm and then drying the radiation shielding material on the first shielding film; a third film forming step of forming a third shielding film by applying at a thickness of 0.1 mm to 0.3 mm and then drying the radiation shielding material on the second shielding film; a fourth film forming step of forming a fourth shielding film by applying at a thickness of 0.1 mm to 0.4 mm and then drying the radiation shielding material on the third shielding film; and a fifth film forming step of forming a fifth shielding film by applying at a thickness of 0.2 mm to 0.45 mm and then drying the radiation shielding solution on the fourth shielding film.

The shielding material applying step may be to apply and laminate sequentially the radiation shielding material on one side of the base material N (4≤N≤8) times so that a total cumulative applying thickness of the radiation shielding material is 0.5 mm to 2.0 mm.

The shielding material applying step may comprise a front film forming step comprising a first applying step of forming a first radiation shielding film by initially applying the radiation shielding material on one side of the base material and a rear film forming step of forming a rear radiation shielding film of at least one layer by additionally applying the radiation shielding material on a front radiation shielding film formed by the front film forming step, wherein the rear film forming step comprises at least one applying step of the radiation shielding material at a different applying thickness as compared with the first applying step.

In an individual applying step of the rear film forming step, the radiation shielding material may be applied thicker than that of the first applying step. The rear film forming step may be sequentially performed a plurality of times and the radiation shielding material may be applied thickest in the last step of the rear film forming step.

The radiation shielding powder may comprise at least one selected from the group consisting of tungsten, bismuth, barium sulfate, antimony, boron, or a compound containing the same. In addition, the binder may comprise at least one selected from the group consisting of a urethane resin, an acrylic resin, an epoxy resin, or a polyester resin.

The multi-layered radiation shielding film may also be formed by the same radiation shielding material containing at least one of the same radiation shielding powder, and at least one layer of the multi-layered radiation shielding film may also contain a different type of radiation shielding powder from another layer of radiation shielding powder.

The radiation shielding material may contain at least one powder of tungsten and a tungsten compound as the radiation shielding powder; and the film laminating step may comprise a shielding material applying step of forming the multi-layered radiation shielding film by sequentially applying the radiation shielding material containing at least one powder of tungsten and a tungsten compound on one side of the base material.

The radiation shielding material may comprise a tungsten shielding material containing at least one powder of tungsten and a tungsten compound as the radiation shielding powder and a bismuth shielding material containing at least one shielding powder of bismuth and a bismuth compound as the radiation shielding powder.

In addition, the film laminating step may comprise a tungsten film forming step of forming at least one layer of the radiation shielding film with the tungsten shielding material, and a bismuth film forming step of forming at least one layer of the radiation shielding film with the bismuth shielding material, before or after the tungsten film forming step.

The tungsten film forming step may comprise a step of forming at least two layers of the radiation shielding film with the tungsten shielding material in a surface contact state; and the bismuth film forming step may be performed before or after the tungsten shielding material applying step and comprise a step of forming at least two layers of the radiation shielding film with the bismuth shielding material in a surface contact state.

The method for manufacturing the lead-free radiation shielding sheet may further comprise a base coating step of forming a base layer for enhancing adhesion of the radiation shielding film on one surface of the base material applied with the radiation shielding material, before the film laminating step.

The base coating step may comprise a step of directly applying the liquid material for forming the base layer on one surface of the base material at a thickness of 0.05 mm to 0.2 mm.

### Advantageous Effects

According to the present invention, the method for manufacturing the lead-free radiation shielding sheet and the lead-free radiation shielding sheet manufacturing by the same have the following effects.

First, according to the present invention, since heavy lead which is harmful to the human body and the environment is not used, side effects such as disease or environmental pollution caused by lead do not occur, the light weight of the radiation shielding sheet is enabled, and protective clothing with excellent wearing sensation can be manufactured, and since flexibility can be improved compared to lead rubber sheets, handling and storage are convenient. In addition, the lead-free radiation shielding sheet manufactured by the present invention can be applied as various designs of clothes and radiation protection means for various uses due to flexibility and ease of operation.

Second, according to the present invention, as compared with another radiation shielding sheet of the same protective performance (radiation shielding performance) using the same material, it is possible to implement a thin film and prevent the sheet from being cracked or broken in a bending environment of the radiation shielding sheet. In addition, even if a separate adhesive or adhesive film is not used, it is possible to secure stable and strong bonding force on an interlayer interface of the radiation shielding film and minimize or prevent occurrence of deviation of the protective performance for each part because the even dispersion of the radiation shielding powder is enabled.

Third, according to the present invention, a single shielding sheet may be used or a plurality of shielding sheets may be overlapped and used to have shielding performance that satisfies a radiation protection standard, and the radiation shielding sheet may be significantly thinner and lighter than lead rubber, and may be applied to various types and designs of radiation protective products, for example, radiation protective clothing, protective wallpapers, protective curtains, protective gloves, protective caps, protective wrapping papers, etc. due to thinning and flexibility.

Fourth, according to the present invention, since a base layer made of an urethane material for stable adhesion of a radiation shielding film is formed on a base material (release paper) having an embossed surface shape and a radiation shielding film of a multilayer structure is formed on the base layer, it is possible to minimize and prevent the deviation in thickness between the layers forming the radiation shielding film, and to stably implement the multi-layered radiation shielding film on the base material.

### DESCRIPTION OF DRAWINGS

Features and advantages of the present invention will be more clearly understood with reference to the drawings to be described below in conjunction with the detailed description of embodiments of the present invention to be described below, in which:

FIG. 1 is a cross-sectional view schematically illustrating an example of a lead-free radiation shielding sheet (protective sheet) manufactured by an embodiment of the present invention;

FIGS. 2A and 2B are process diagrams schematically illustrating a method for manufacturing a lead-free radiation shielding sheet (protective sheet) according to an embodiment of the present invention;

FIG. 3 is an enlarged photograph of the surface of a base material (release paper) having an embossed surface;

FIG. 4 is a diagram schematically illustrating a 3 roll mill;

FIG. 5 is a diagram schematically illustrating a process of milling/dispersing particles by a 3 roll mill;

FIG. 6 is an enlarged photograph showing a bismuth-based radiation shielding powder (bismuth oxide powder);

FIG. 7 is an enlarged photograph showing a tungsten-based radiation shielding powder (tungsten metal powder);

FIGS. 8A and 8B are cross-sectional enlarged photographs showing examples of radiation shielding sheets made of a bismuth powder and a tungsten powder, respectively;

FIGS. 9A and 9B are cross-sectional enlarged photographs showing examples of a radiation shielding sheet comprising both a radiation shielding film made of a bismuth powder and a radiation shielding film made of a tungsten powder;

FIG. 10 is a cross-sectional enlarged photograph of a radiation shielding sheet according to Comparative Example of the present invention; and

FIG. 11 is a diagram illustrating a shielding performance test position of a radiation shielding sheet.

### BEST MODE

Hereinafter, preferred embodiments of the present invention, of which objects of the present invention may be realized in detail, will be described with reference to the accompanying drawings. In describing the embodiments of the present invention, like names and like reference numerals will be used for like configurations and detailed description for known techniques will be omitted below.

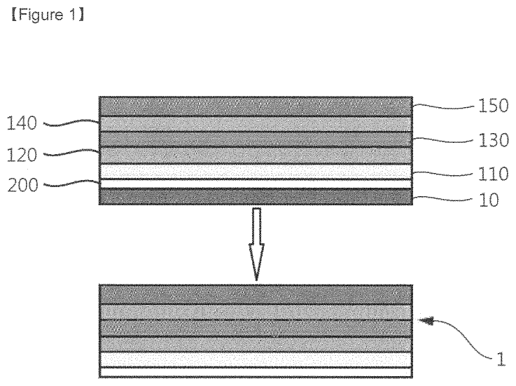

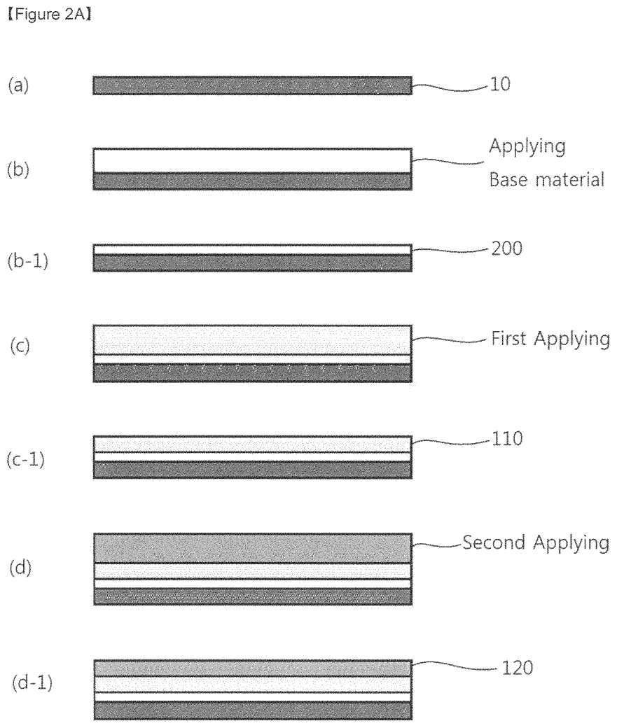

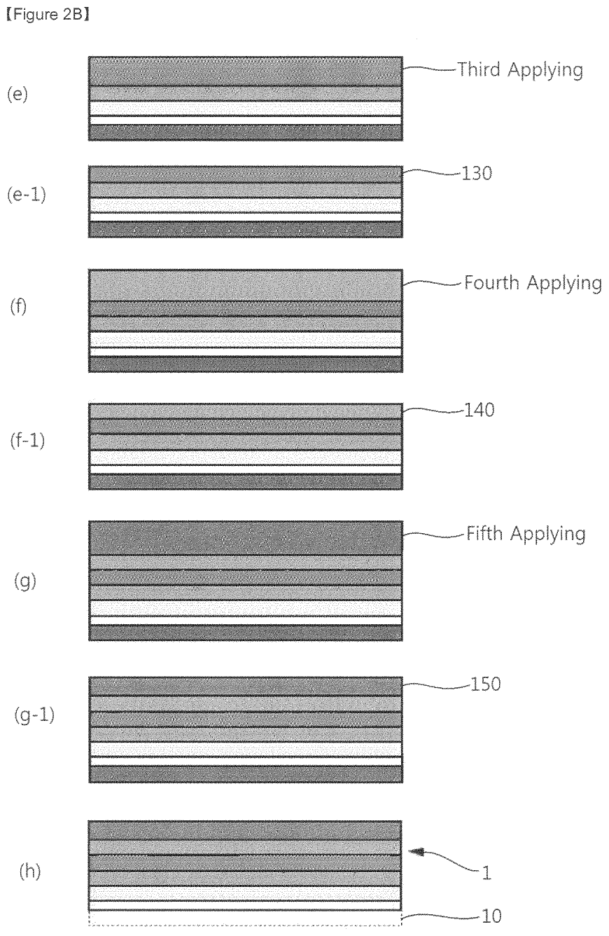

First, a method for manufacturing a radiation shielding sheet according to an embodiment of the present invention and an embodiment of a radiation shielding sheet 1 (protective sheet) will be described with reference to FIGS. 1 and 2.

The radiation shielding sheet 1 manufactured by an embodiment of the present invention is a radiation shielding sheet without containing a lead component, that is, a lead-free radiation shielding sheet, and is a flexible radiation shielding material for shielding a radiation such as X-rays.

The method for manufacturing the lead-free radiation shielding sheet according to an embodiment of the present invention (hereinafter, referred to as ‘a method for manufacturing a protective sheet) comprises a film laminating step (steps (c) to (g-1) of FIGS. 2A and 2B) of forming a multi-layered radiation shielding film 100 on one side of a base material 10 by repeating a process of sequentially applying, drying, and integrating a radiation shielding material containing a radiation shielding powder and a binder for forming a film, for example, a polymer resin to be mixed with each other on one side of the base material 10.

An example of the radiation shielding material applicable to the method for manufacturing the protective sheet according to the present embodiment may include a liquid material containing a binder resin, a solvent, and a powder containing a radiation shielding metal powder other than lead (Pb), that is, a radiation shielding solution. Accordingly, when the liquid radiation shielding material is applied on one side of the base material 10, a film, that is, a film is formed with the binder resin while the solvent is evaporated.

The multi-layered radiation shielding film 100 may also be applied/coated directly on the surface of the base material 10, but as described below, may also be indirectly coated on the surface of the base material 10 via another layer, for example, a resin layer (a base layer in the present embodiment) without containing the radiation shielding powder.

The method for manufacturing the protective sheet according to the present embodiment may further comprise a base coating step (steps (b) to (b-1) of FIG. 2A) of forming a resin layer 200 (hereinafter, referred to as ‘a base layer’) on one surface of the base material applied with the radiation shielding material, before the film laminating step.

The base coating step is a step of forming the resin layer, that is, the base layer 200 directly on the surface of the base material 10 by applying (step (b) of FIG. 2A, applying of the base material) and heat-drying, on the surface of the base material 10, a liquid resin composition containing a polymer, as a more specific example, a resin such as an urethane resin, an acrylic resin, an epoxy resin, or a polyester resin, and a solvent, that is, a liquid material (base material) for forming the base layer.

In the present embodiment, the aforementioned urethane resin is applied as a resin for forming the base layer, but it is natural that the type of resin for forming the base layer is not limited thereto, and the base layer 200 enhances the adhesion of the radiation shielding film 100 to prevent separation and peeling of the radiation shielding film 100.

In the present embodiment, the base material 10 is a sheet constituting a flooring material for forming the radiation shielding sheet, and may be a fabric such as a textile, knitted fabric, or a nonwoven fabric, but in the present embodiment, in order to stably implement the multi-layered radiation shielding film 100, a release paper is exemplified as the base material 10.

More specifically, the base material 10 is a release paper that has an embossed surface having an embossed shape and can be separated from the base layer 200, that is, an embossed release paper. In addition, the base applying step is a step of forming the base layer by applying and then heat-drying the liquid material (hereinafter, referred to as ‘the urethane solution’) for forming the base layer on the surface of the release paper, that is, the base material 10 at a predetermined thickness.

Accordingly, an embodiment of the present invention, as a method for manufacturing the protective sheet of manufacturing a lead-free radiation shielding sheet including the base layer 200 made of an urethane resin and the multi-layered radiation shielding film 100 coated (laminated) on the base layer, comprises a base coating step of forming the base layer 200 by applying and heat-drying an urethane solution containing an urethane resin and a solvent on the surface of the base material 10, for example, the aforementioned release paper, and a shielding film forming step of laminating and forming the multi-layered radiation shielding film 100 on the base layer 200 by repeating a process of applying and drying a radiation shielding solution containing an urethane resin, a solvent, and a bismuth powder on the base layer 200 a plurality of times.

The base coating step comprises a step of applying the urethane solution, that is, the liquid material for forming the base layer on the surface of the base material 10 at a thickness of 0.05 mm to 0.2 mm, for example, a thickness of 0.08 mm to 0.18 mm, more specifically a thickness of 0.1 mm to 0.15 mm. That is, in order to form the base layer 200 on the surface of the base material 10, the urethane solution (base material) is applied on the surface of the base material, that is, the release paper at the aforementioned thickness ((b) of FIG. 2A), and when the solvent contained in the urethane solution is s radiated (evaporated) by drying, for example, a heat drying method, the thickness of the urethane solution is reduced ((b-1) of FIG. 2A) and then the base layer 200 made of the urethane resin is formed on the surface of the release paper 10.

The base layer 200 stably bonds a radiation shielding material dispersing and containing the radiation shielding powder, more specifically the radiation shielding film 100 to the release paper 10, and helps to transfer the surface curved state of the release paper 100 to an interlayer interface of the radiation shielding film 100 having the multi-layered structure. Therefore, the interlayer bonding force of the lead-free radiation shielding sheet 1 according to the present embodiment may be enhanced.

When forming the base layer 200, if the applying thickness of the liquid material for forming the base layer is less than 0.05 mm, coating workability deteriorates and the radiation shielding film 100 is not stably fixed, and if the applying thickness is more than 0.2 mm, a thickness deviation may occur for each part in the base layer, affects the thickness of the radiation shielding sheet, and interferes with the smooth evaporation of the solvent.

More specifically, when considering aspects of the coating workability of the base layer, the fixing force of the radiation shielding film 100, resolution of the thickness deviation for each part in the base layer, and the smooth evaporation of the solvent, the liquid material for forming the base layer, that is, the urethane solution may be applied on the base material 10, that is, the release paper at a thickness of 0.08 mm to 0.18 mm, preferably 0.1 mm to 0.15 mm.

The urethane solution for forming the base layer 200 is contained at 50 to 70 parts by weight of the solvent, more specifically 55 to 65 parts by weight, based on 100 parts by weight of the urethane resin, but is not limited thereto. The solvent includes dimethylformamide (DMF), isopropyl alcohol (IPA), methyl ethyl ketone (MEK), toluene, etc., and these solvents may be mixed alone or in combination to be used as the above-described solvent.

In order to form the base layer 200, the urethane solution of approximately 2,000 to 2,500 cps may be applied on the release paper 10, but the viscosity of the urethane solution is not limited thereto, and may be changed according to process conditions. For example, the viscosity of the urethane solution for the base layer, that is, the liquid material for forming the base layer may be adjusted by mixing the above-described solvent to the urethane resin at about 50,000 to 80,000 cps.

When using the embossed release paper described above as the base sheet 10, there are the following advantages.

First, the surface having an embossed shape, that is, the embossed surface may minimize a flowing phenomenon flowing from the surface of the base material when the urethane solution is applied on the surface of the base material 10 (release paper) at the thickness and induce uniform applying of the radiation shielding material so as to minimize the thickness deviation for each part of the radiation shielding material applied on the base layer 200.

Second, while the urethane solution is applied on the surface of the base material, the urethane solution is embedded in an uneven structure to prevent the base layer and the radiation shielding film from being processed to be thinner than the thickness on a process design, thereby minimizing insufficient radiation shielding performance or the occurrence of a deviation for each part.

Third, physical properties may be given so as to vary the applying layer according to the mass and viscosity of the radiation shielding material upon additional lamination of the radiation shielding material by maintaining coating stability so that the liquid radiation shielding material is evenly applied well in the process of forming the multi-layered radiation shielding film and inducing the smooth evaporation of the solvent in the process of heat-drying the radiation shielding material.

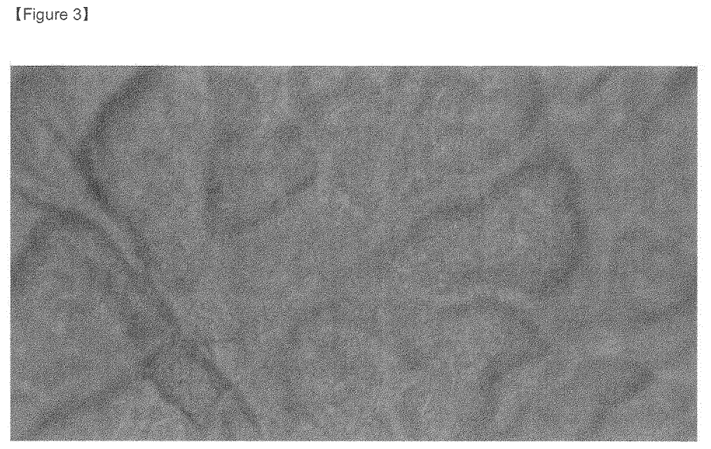

An example of the embossed release paper may include a DN-TP release paper (Ajinomoto Co., Ltd., Non-silicon type release paper developed by Dai Nippon Printing Co., Ltd.), and in FIG. 3, a surface enlarged photograph of the DN-TP release paper is illustrated.

Next, the film laminating step comprises a shielding material applying step of applying and drying sequentially the radiation shielding material on one side of the base material 10, more specifically, on the base layer 200 a plurality of times, so that the radiation shielding film 100 has a multilayer structure of at least three layers.

In the shielding material applying step, an N-layered radiation shielding film 100 may be formed by sequentially applying and drying the radiation shielding material on one side of the base material 10 N (3≤N≤10) times.

As a specific example, the film laminating step may comprise a shielding material applying step of repeating sequentially the process of applying at a thickness of 0.05 mm to 0.50 mm and drying the radiation shielding material on one side of the base material 10 a plurality of times, more specifically 0.08 mm to 0.40 mm per one applying (applying of the radiation shielding material of forming a single-layered shielding film).

The shielding material applying step may also comprise a film forming step of performing the process of sequentially applying at a thickness of 0.1 mm to 0.30 mm and drying the radiation shielding material on one side of the base material 10 at least two times.

In the present embodiment, the film forming step (steps (c) to (f-1) of FIG. 2A/2B) is an inner film forming step, that is, a step of forming a shielding film of at least two layers of inner shielding films 110 to 140 formed before forming a layer 150 (surface shielding film) forming the last radiation shielding film, that is, the surface of the radiation shielding film 100 which is laminated/formed last in the film laminating step.

In the shielding material applying step, an N-layered radiation shielding film 100 is formed by sequentially laminating and applying the radiation shielding material on one side of the base material 10 N (4≤N≤8) times so that a total cumulative applying thickness of the liquid radiation shielding material applied on one side of the base material 10, more specifically the base layer 200 is 0.5 mm to 2.0 mm. The present embodiment is an example of forming a five-layered radiation shielding film, but of course, the layer number of the radiation shielding film is not limited thereto. For example, by performing a shielding material applying step of laminating the radiation shielding material 5 times at a total cumulative applying thickness of 0.6 mm to 1.75 mm, a radiation shielding film having a 5-layered structure may be formed on one side of the base material.

The shielding material applying step may comprise a front film forming step (steps (c) to (d-1) of FIG. 2A) comprising a first applying step (steps (c) to (c-1) of FIG. 2A) of forming a first radiation shielding film 110 (first shielding film) by initially applying the radiation shielding material on one side of the base material, and a rear film forming step (steps (e) to (g-1) of FIG. 2B) of forming rear radiation shielding films 130, 140, and 150 of at least one layer by additionally applying the radiation shielding material on the surface of the front radiation shielding films 110 and 120 of at least one layer formed by the front film forming step.

The rear film forming step (steps (e) to (g-1) of FIG. 2B) may comprise at least one applying step of applying the radiation shielding material at a different thickness when comparing the first applying step (steps (c) to (c-1) of FIG. 2A).

In an individual applying step of the rear film forming step, the radiation shielding material may be applied to be thicker than that of the first applying step. In the rear film forming step, the process of applying and drying the radiation shielding material is sequentially performed a plurality of times, and in the last applying step (steps (g) to (g-1) of FIG. 2B) of forming a surface layer 150 of the radiation shielding film in the rear film forming step, the radiation shielding material may be applied thickest.

The radiation shielding powder may include at least one selected from the group consisting of tungsten, bismuth, barium sulfate, antimony, boron, or a compound containing the same (a material containing any one of tungsten to boron as an element of the compound). In other words, the radiation shielding material may include one or more radiation shielding powders selected from the group consisting of tungsten, bismuth, barium sulfate, antimony, boron, or a compound containing the same (a material containing any one of tungsten to boron as an element of the compound). Accordingly, a single type of radiation shielding powder may be contained in a single-layered shielding film, and two or more types of radiation shielding powders may be contained.

In addition, the binder, that is, a resin made of a polymer may include at least one selected from the group consisting of an urethane resin, an acrylic resin, an epoxy resin, or a polyester resin. Of course, the types of the radiation shielding powder and the binder are not limited to the examples described above. As described in the aforementioned base layer, the solvent of the radiation shielding material may use dimethylformamide (DMF), isopropyl alcohol (IPA), methyl ethyl ketone (MEK), toluene, etc., and these solvents may be used alone or in combination as the above-described solvent.

Based on 100 wt % of the above-mentioned radiation shielding material, a liquid radiation shielding material containing 20 to 45 wt % of the binder (resin), 15 to 30 wt % of the solvent, and 35 to 60 wt % of the radiation shielding powder may be used, but it is natural that the content of each component is not limited thereto. For example, when tungsten (including a tungsten compound) is applied as the radiation shielding powder, it is preferable that the content of the radiation shielding powder is 45 wt % or less based on 100 wt % of the radiation shielding material for uniform dispersion and adhesion stability of the radiation shielding powder in the shielding film.

As a specific example, based on 100 wt % of the radiation shielding material, the urethane resin of 20 to 45 wt %, more specifically 25 to 40 wt %, the solvent of 15 to 30 wt %, more specifically 15 to 25 wt %, and the radiation shielding powder such as bismuth or tungsten of 35 to 60 wt %, more specifically 40 to 55 wt % may be included, but are not limited thereto, and may be variously changed in a range that can be applied and dried, and it is natural that additives such as a dispersing agent may be contained. The content of each component may be adjusted, and for example, when tungsten (including a tungsten compound) is applied as the radiation shielding powder, it is preferable that the content of the radiation shielding powder is 45 wt % or less based on 100 wt % of the radiation shielding material for uniform dispersion and adhesion stability of the radiation shielding powder (tungsten powder) in the shielding film.

The radiation shielding solution for forming the above-mentioned radiation shielding film 100 may include 30 to 38 wt % of the urethane resin, 15 to 27 wt % of the solvent, and 40 to 50 wt % of the bismuth powder. More specifically, based on 100 wt % of the radiation shielding solution, 32 to 36 wt % of the urethane resin, 18 to 24 wt % of the solvent, and 43 to 47 wt % of the bismuth powder may be included.

When describing a more specific example of the laminating and applying of the radiation shielding material, the shielding material applying step may also comprise a first film forming step of forming a first shielding film 110 by applying (first applying) at a thickness of 0.1 mm to 0.3 mm and drying the radiation shielding material on one side of the base material 10, that is, the base layer 200 in the present embodiment, a second film forming step of forming a second shielding film 120 by applying (second applying) at a thickness of 0.1 mm to 0.3 mm and then drying the radiation shielding material on the first shielding film 110, a third film forming step of forming a third shielding film 130 by applying (third applying) at a thickness of 0.1 mm to 0.3 mm and then drying the radiation shielding material on the second shielding film 120, a fourth film forming step of forming a fourth shielding film 140 by applying (fourth applying) at a thickness of 0.1 mm to 0.4 mm and then drying the radiation shielding material on the third shielding film 130, and a fifth film forming step of forming a fifth shielding film 150 by applying (fifth applying) at a thickness of 0.2 mm to 0.45 mm and then drying the radiation shielding material on the fourth shielding film 140.

In the present embodiment, the fifth shielding film 150 forms the above-described surface shielding film, that is, a surface layer of the radiation shielding sheet according to the present embodiment.

All layers of the multi-layered radiation shielding film may be formed of the same radiation shielding material, and the multi-layered radiation shielding film may include layers containing different types of radiation shielding powders.

For example, all the layers of the multi-layered radiation shielding film may be formed by applying/drying the radiation shielding material containing the same type of radiation shielding powder, as a specific example, a bismuth powder or a tungsten powder.

As a more specific example, the radiation shielding material may contain a tungsten powder as the radiation shielding powder. In this case, the film laminating step comprises a shielding material applying step of forming the multi-layered radiation shielding film by sequentially applying the radiation shielding material containing the tungsten powder on one side of the base material 10.

The tungsten powder is a concept of including not only a tungsten metal powder but also a compound (tungsten compound) containing tungsten as an element of the compound, for example, a carbide tungsten powder such as a tungsten carbide powder.

As another example, the radiation shielding material may contain the aforementioned bismuth powder as the radiation shielding powder. In this case, the film laminating step comprises a shielding material applying step of forming the multi-layered radiation shielding film by sequentially applying the radiation shielding material containing the bismuth powder on one side of the base material 10.

The bismuth powder is also a concept of including a bismuth metal powder (pure bismuth powder) or a compound thereof (a material containing bismuth as an element of the compound), for example, a bismuth compound such as bismuth oxide.

Examples of the bismuth oxide include bismuth trioxide (Bi₂O₃), sodium bismuthate (BiNaO₃), bismuth nitrate (BiN₃O₉), etc., which may be used alone or in combination as the bismuth powder.

As described above, by applying/drying the same type of radiation shielding material on one side of the base material 10, a radiation shielding sheet having a structure in which all the layers of the radiation shielding film contain the same radiation shielding powder may be manufactured.

As another example, the multi-layered radiation shielding film may also include a shielding film formed by applying/drying the radiation shielding material containing the bismuth powder as the radiation shielding powder and a shielding film formed by applying/drying the radiation shielding material containing the tungsten powder as the radiation shielding powder.

More specifically, the radiation shielding material includes a first shielding material containing the tungsten powder as the radiation shielding powder, and a second shielding material containing the bismuth powder as the radiation shielding powder.

In other words, a plurality of types of radiation shielding materials containing different types of radiation shielding powders may be used in the manufacturing of the radiation shielding film. The first shielding material is a radiation shielding material (tungsten shielding material) containing a tungsten powder (at least one powder of tungsten or a tungsten compound), and the second shielding material is a radiation shielding material (bismuth shielding material) containing a bismuth powder (at least one powder of bismuth or a bismuth compound). That is, the shielding film of each layer may be formed of any one radiation shielding material selected from the group consisting of the tungsten shielding material and the bismuth shielding material.

In addition, the film laminating step may comprise a tungsten film forming step of forming at least one layer of the radiation shielding film with the tungsten shielding material and a bismuth film forming step of forming at least one layer of the radiation shielding film with the bismuth shielding material, before or after the tungsten film forming step.

Therefore, the shielding material applying step comprises the tungsten film forming step and the bismuth film forming step described above. In addition, the multi-layered radiation shielding film may include a shielding film (a tungsten film; hereinafter referred to as a ‘W film’) formed of a tungsten shielding material and a shielding film (a bismuth film; hereinafter referred to as a ‘B film’) formed of a bismuth shielding material.

For example, a radiation shielding film may also be manufactured with a structure in which two or more layers of W film are continuously laminated and then two or more layers of B film are continuously laminated. In addition, a radiation shielding film may also be manufactured with a structure in which two or more layers of B film are continuously laminated and then two or more layers of W film are continuously laminated. Also, a radiation shielding film may be manufactured with a structure in which at least one layer of B film and at least one layer of W film are alternately laminated.

Accordingly, the W film forming step may comprise a step of forming at least two layers of the radiation shielding film in a surface contact state (continuously laminated state) with the first shielding material (tungsten shielding material). In addition, the B film forming step is performed before or after the W film forming step and may comprise a step of forming at least two layers of the radiation shielding film in a surface contact state with the second shielding material (bismuth shielding material).

More specifically, the shielding material applying step according to the present embodiment may comprise the front film forming step and the rear film forming step as the aforementioned example. In the front film forming step, two layers or more of radiation shielding film (W film), for example, two layers of W film may be continuously formed by applying/drying the tungsten shielding material on one side of the base material 10. In the rear film forming step, two layers or more, for example, three layers of radiation shielding film (B film) may be continuously formed by applying/drying the bismuth shielding material on the surface of the multi-layered W film. In this case, the first radiation shielding film may be formed of the tungsten shielding material.

Unlike this, in the front film forming step, two layers or more, for example, two layers of radiation shielding film (B film) may be continuously formed by applying/drying the bismuth shielding material on one side of the base material 10. In the rear film forming step, two layers or more, for example, three layers of radiation shielding film (W film) may be continuously formed by applying/drying the tungsten shielding material on the surface of the multi-layered B film. In addition, before the aforementioned front film forming step, the above-described base coating step, that is, the step of forming the base layer 200 on the surface of the base material 10 may be performed. In addition, an aging process may be performed between the front film forming step and the rear film forming step to remove an effect on heat.

In the present embodiment and the drawings, a lead-free protective shielding sheet having a 5-layered radiation shielding film 100 and a single-layered base layer 200 has been disclosed, but it is natural that the number of layers of the radiation shielding film is not limited thereto. The present invention discloses a lead-free radiation shielding sheet comprising a multi-layered radiation shielding film 100 by continuously laminating (applying/drying) one or multiple types of radiation shielding materials. Then, after the radiation shielding film 100 is formed into a multilayer in various methods described above, the base material 10 is peeled off and removed.

In the embodiments of the present invention, as compared with a single-layered radiation shielding film manufactured by a process of applying and then drying a radiation shielding material in a single layer by the same thickness as the total cumulative applying thickness of the radiation shielding material applied sequentially a plurality of times on one side of the base material to form the radiation shielding film having the multi-layered structure, it is possible to smooth the curing (evaporation of the solvent) of the radiation shielding film 100, implement tissue stability and interfacial bonding stability of the radiation shielding film 100, improve a radiation shielding effect because the radiation shielding powder is evenly dispersed, and minimize the thickness of the radiation shielding film 100 while maintaining the tissue stability.

The radiation shielding material for forming the above-mentioned radiation shielding film 100 is a material having fluidity as described above, that is, a liquid material, and in an embodiment to be described below, the radiation shielding material includes 25 to 40 wt % of the urethane resin as the binder described above, 15 to 25 wt % of the solvent such as DMF, MEK, and toluene, and 40 to 55 wt % of the bismuth powder or the tungsten powder.

The viscosity of the urethane solution applied for forming the base layer and the viscosity of the radiation shielding material applied step by step for forming the shielding film may be appropriately adjusted according to conditions such as particle size and shape of the radiation shielding powder and an environment for forming the film, and a method for adjusting the viscosity is known, and thus additional description will be omitted.

The above-mentioned shielding films 110, 120, 130, 140, and 150 may be formed by the radiation shielding solution having the same component/content as described above, and at least one of the shielding films 110, 120, 130, 140, and 150 may be formed by a radiation shielding material having a different content of at least one component or a different type of radiation shielding powder within a range illustrated above. For example, even if all the layers of the radiation shielding film are formed on the radiation shielding material containing the same type of radiation shielding powder, the content ratio of bismuth or tungsten powder may be applied differently for each layer.

On the other hand, the urethane resin is a binder, the polyurethane resin is excellent in the surface adhesion (bonding force) of the base material 10, such as a fiber material or the release paper described above, high in durability and excellent in flexibility to be suitable as a shielding material, and high in hydrogen density to reduce high-speed neutrons. The urethane resin, that is, the polyurethane resin itself, the manufacturing method thereof, etc. are known, and thus the additional description thereof will be omitted.

In addition, the drying of the urethane solution applied to form the base layer 200 and the drying of the radiation shielding material applied step by step to form the shielding film may be performed for 40 sec to 70 sec by a heat drying method (hot drying method) in a heat drier (heat dry oven) of 100° C. to 130° C. However, a drying method such as a drying temperature and a drying time is not limited thereto, and may be variously changed under conditions capable of implementing a predetermined drying state, and the drying time and/or temperature may be lowered under drying conditions in which drying air flows.

For example, while a strip type long base sheet 10 (releasing paper) is continuously transferred by a roller, when the base layer 200 and the radiation shielding film 100 are laminated/formed thereon, in a heat drying environment of 115° C. to 130° C., it is possible to pass a heat dryer (heat drying chamber) having a length of approximately 15 m to 30 m at a predetermined speed that can be cured, for example, a speed of 10 to 35 m per minute, specifically 10 m to 18 m.

As a more specific example, the evaporation of the solvent, that is, the heat drying may be performed in the same manner as that the first drying is performed while a portion applied with the urethane solution passes through a heat drier of 17 m to form the base layer 200, the second drying is performed while a portion applied with the radiation shielding solution passes through a heat drier of 22 m in a first step to form the first shielding film 110 directly laminated on the base layer 200, and the third drying is performed while a portion applied with the radiation shielding solution passes through a heat drier of 25 m in a second step to form the second shielding film 120 directly laminated on the first shielding film. In addition, as described above, after the base layer 200, the first shielding film 110, and the second shielding film 120 are formed sequentially, the process of continuously laminating/forming the third shielding film 130, the fourth shielding film 140, and the fifth shielding film 150 on the second shielding film 120 in sequence may also be subjected to the same process as the process of forming the base layer, the first shielding film, and the second shielding film described above. However, the above-described heat drying environment, that is, a heating temperature, a transfer speed, and a length of a heat drying period may be variously changed within a range capable of sufficient heat drying.

In addition, in the radiation shielding material for forming the multi-layered radiation shielding film 100, the bismuth powder and the tungsten powder, fine particles having an average size (r) of 0<r≤5 μm, more specifically granular bismuth nanoparticles having a size of up to 1,000 nm (1 μm), for example 10 nm≤r≤1 μm, are preferable for even dispersion in the urethane resin. However, as the sizes of the bismuth or tungsten particles are milled to be small, high cost may be required in manufacturing, and thus, considering the cost aspect of manufacturing, the radiation shielding powder may be used in the range of at least 50 nm to 100 nm, and the powder of up to 1000 nm or less, more specifically 100 nm to 1000 nm.

The method for manufacturing the lead-free radiation shielding sheet according to the present embodiment may further comprise a milling step of performing dispersion and milling of the radiation shielding powder and even mixing with the resin by milling a shielding raw material composition containing the binder resin such as the urethane resin and the radiation shielding powder such as the bismuth powder or tungsten powder, for manufacturing the radiation shielding material.

As the bismuth powder contained in the raw material composition, fine particles having an average particle size of 0.1 μm to 6 μm, more specifically 0.5 μm to 2 μm may be applied, but the size is not limited thereto. In addition, as the tungsten powder contained in the raw material composition, fine particles having an average particle size of 0.1 μm to 2 μm, more specifically 0.1 vim to 1 μm are used, but the size is not limited thereto.

The particle size and shape of the radiation shielding powder, such as the bismuth powder or the tungsten powder, may act as important factors for reducing a deviation in radiation shielding performance for each site with the uniform dispersion ability of the powder when mixed with a resin used as a substrate, for example, an urethane resin.

Thus, fine-sized powders, for example, micro-particles or nano-particles obtain an entirely uniform shielding effect by milling the bismuth powder or tungsten powder and dispersing the milled bismuth powder or tungsten powder evenly in the resin using a milling device, for example, a 3 roll mill.

The shielding raw material composition (the composition supplied to milling) described above in the present embodiment is a liquid material in which the urethane resin, the bismuth or tungsten powder, and the solvent are mixed, and has the viscosity of about 2,000 to 2,500 cps, but is not limited thereto and may be changed by forming conditions of the radiation shielding film, for example, a transfer speed or heat drying conditions of the base material 10. The composition ratio of the shielding raw material composition may be the same as that of the radiation shielding material, or the content of the solvent may be slightly increased compared to the radiation shielding material when considering partial evaporation of the solvent during milling.

Therefore, in the present embodiment, assuming that there is no loss of each component in the milling process, the composition (shielding raw material composition) before milling and the composition (radiation shielding material) after milling are materials of the same component and content, but the composition after milling, that is, the radiation shielding material is a material in which the radiation shielding powder is evenly dispersed in a resin (binder).

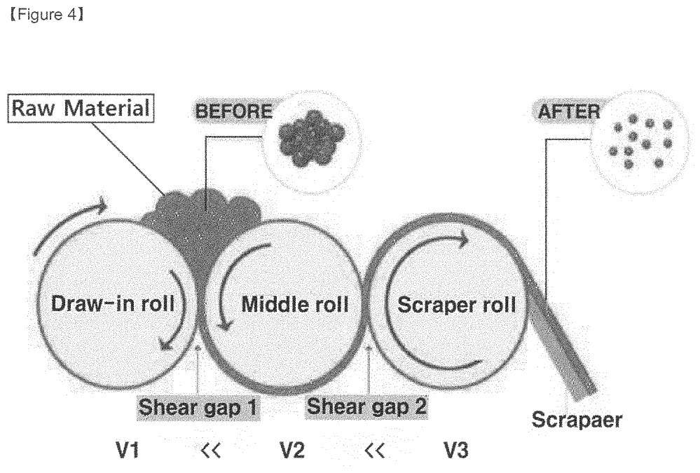

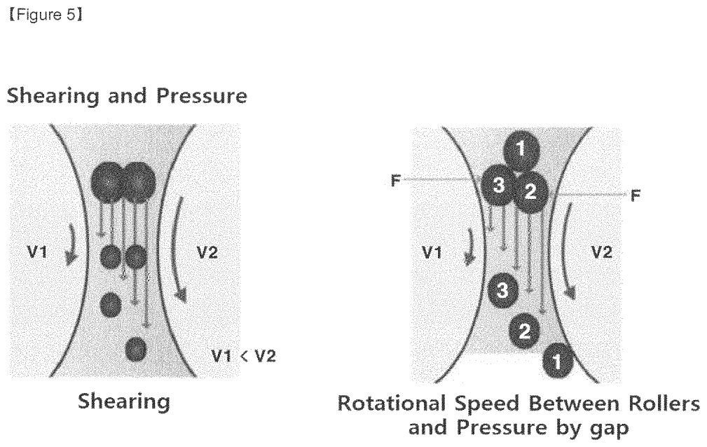

When describing in more detail with reference to FIGS. 4 and 5, the composition containing the binder (resin), the solvent and the radiation shielding powder, that is, the above-mentioned shielding raw material composition is milled to uniformly mix/disperse the radiation shielding powder and the resin and mill the radiation shielding powder.

In the milling process by the 3 roll mill, when a paste of high-viscosity urethane resin passes between three rollers rotating at different rotational speeds to have optimized density and stiffness, rubbing occurs due to a difference in rotational speed between the rollers to obtain an effect of precise milling and dispersion.

In the above-mentioned 3 roll mill, each roller rotates at a constant rate of rotational speed (rpm) to apply pressure and shear force to a sample to enable mixing, milling, and dispersion described above. Through this, a colloidal radiation shielding material similar to a colloidal state may be implemented to reduce the sizes of bismuth particles and tungsten particles and maintain a uniform dispersion state without the precipitation of the radiation shielding powder in the urethane resin by the gravity.

For reference, the 3 roll mill is a structure in which three rolls rotating at different speeds V1, V2, and V3 in opposite directions to each other are horizontally disposed in parallel and is a principle in which the sample (shielding raw material composition) passes between a middle roll and a first roll (draw-in roll) to be transferred to the last roll (scraper roll), and a dispersed sample is discharged by a scraper through the last roll (scraper roll). The milling device itself, which disperses the particles evenly in the resin, such as a 3 roll mill, is well known, and thus additional description thereof will be omitted.

The embodiment of the lead-free radiation shielding sheet, that is, the lead-free protective sheet according to the present invention may comprise a base layer 200 made of an urethane resin coated on the surface of the release paper 10 having an embossed surface having an embossed shape and a multi-layered radiation shielding film 100 having a plurality of shielding films 110, 120, 130, 140, and 150 which are continuously laminated on the base layer 200 sequentially. In addition, the shielding films each includes an urethane resin and a bismuth powder or tungsten powder, and more specifically, 80 to 200 parts by weight of the bismuth powder or tungsten powder with respect to 100 parts by weight of the binder, that is, the urethane resin.

In addition, the radiation shielding film 100 as the multi-layered thin film shielding layer as described above is a 5-layered film comprising a first shielding film 110 formed on the base layer 200, a second shielding film 120 formed on the first shielding film, a third shielding film 130 formed on the second shielding film, a forth shielding film 140 formed on the third shielding film, and a fifth shielding film 150 formed on the fourth shielding film. However, in FIGS. 1, 2A, and 2B, although the boundaries between the shielding films are divided, actually, in the method of sequentially laminating the same radiation shielding material, the boundaries between the shielding films formed by the same radiation shielding material may not be clearly divided. The radiation shielding material applied on the first generated shielding film fills pinholes of the first generated shielding film and is cured by the evaporation of the solvent, so that the shielding films may be seen as a single film without a clearly divided boundary. Of course, when different types of radiation shielding materials including radiation shielding powders of different colors are alternately applied, the shielding films may be identified by the colors of the radiation shielding powders.

In a step of forming the last second or third film applied to form shielding films of two layers or three layers formed last in the aforementioned radiation shielding film, respectively, a applying thickness of the radiation shielding material to be applied may be set to 2.0 mm to 4.0 mm, but is not limited thereto.

In addition, in the step of forming the film of each order performed before the step of forming the last second or third film having the applying thickness of 2.0 mm to 4.0 mm described above in the aforementioned radiation shielding film, the applying thickness of the radiation shielding material may be set to 0.1 mm to 2.5 mm, but is not limited thereto.

The present embodiment may provide a method for manufacturing a lead-free radiation shielding sheet capable of implementing 80 to 90% of the thickness shrinkage of the final radiation shielding film compared to the total cumulative applying thickness of the radiation shielding material, that is, a method for manufacturing a lead-free radiation shielding sheet of forming a multi-layered radiation shielding film at a thickness of 1/10 to ⅕ smaller than the total cumulative applying thickness of the radiation shielding material.

By the above-mentioned 3 roll mill, the radiation shielding powder may be milled smaller than the size before milling, and a rotation ratio (V1:V2:V3) of the first roll (draw-in roll), the middle roll, and the last roll (scraper roll) is 1:2:3, but is not limited thereto, of course.

The above-mentioned radiation shielding sheet 1 may be applied to the manufacture of protective clothing, that is, radiation shielding clothing or hats or gloves, and for example, the radiation shielding sheet is embedded (buried) in a surface applying material (fiber) to implement the radiation protection. The radiation shielding sheet 1 may be used with one sheet or applied with a plurality of sheets to be overlapped in accordance with the required protection performance. The radiation shielding sheet is a flexible thin film sheet and may be used for various purposes such as wallpapers, floorings, or wrapping papers.

The radiation shielding sheet 1 may be fixed to a garment for protective clothing by a method such as sewing or adhesion. In addition, a plurality of radiation shielding sheets 1 may be integrated in an overlapped state by sewing or adhesion.

According to the above-described embodiment, it is possible to manufacture the radiation shielding sheet 1 which has an excellent radiation shielding effect, is easily recycled and eco-friendly compared to lead, and is excellent in light weight and flexibility.

Hereinafter, the configuration and operation of the present invention will be described in more detail through specific Examples of the present invention. However, the following Examples are only illustrative to help the understanding of the present invention, and the scope of the present invention is not limited to the following Examples. In addition, descriptions of contents that can be sufficiently known or inferred through known techniques by those skilled in the art will be omitted.

### 1. Preparation of Radiation Shielding Material

For the preparation of a radiation shielding material, two types of liquid radiation shielding materials were obtained using two types of shielding raw material compositions (samples).

The content ratio of a binder, a solvent, and a radiation shielding powder used for the shielding raw material composition was 30 wt % of an urethane resin (binder), 20 wt % of a solvent, and 50 wt % of a bismuth powder or tungsten powder.

In addition, in a 3 roll mill device, rotational speeds of a first roll (draw-in roll), a middle roll, and a last roll (scraper roll) were 500 RPM, 1,000 RPM, and 1,500 RPM, respectively, and a gap between the rolls was 10 μm or less, and was about 5 μm.

### Example 1 of Radiation Shielding Material

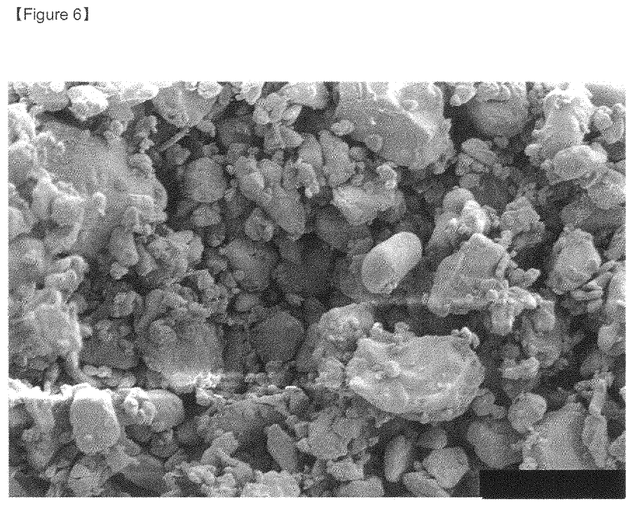

In order to prepare a radiation shielding material according to Example 1, as illustrated in FIG. 6, a commercial bismuth powder (Bi₂O₃, Changsha Santech Materials Co., Ltd, in China) having an average size of 0.5 μm to 2 μm and commercially available urethane resin and solvent were used. A shielding raw material composition containing a bismuth powder having a size of 0.5 μm to 2 μm, an urethane resin and a solvent (DMF/MEK) in the above-described content ratio was milled with a 3 roll mill device to obtain a liquid radiation shielding material according to Example 1, that is, a bismuth shielding material.

### Example 2 of Radiation Shielding Material

In order to prepare a radiation shielding material according to Example 2, as illustrated in FIG. 7, a commercial tungsten powder (tungsten metal powder, TaeguTec LTD, in Korea) having an average size of 0.2 μm to 0.5 μm was used. A shielding raw material composition containing a tungsten powder having a size of 0.2 μm to 0.5 μm, an urethane resin and a solvent (DMF/MEK) in the above-described content ratio was milled with a 3 roll mill device to obtain a liquid radiation shielding material according to Example 2, that is, a tungsten shielding material.

In the radiation shielding materials according to Examples 1 and 2, the radiation shielding powders (the bismuth powder and the tungsten powder) were entirely evenly dispersed and gelatinized like colloids without precipitation.

### 2. Preparation of Examples and Comparative Example of Radiation Shielding Sheets

Examples 1 to 4 and Comparative Example of radiation shielding sheets according to the present invention were prepared as follows by using an embossed release paper having a size of 1 m×1 m (width×length) (DN-TP release paper, Ajinomoto Co., Ltd., Non-silicon type release paper developed by Dai Nippon Printing Co., Ltd.).

### Example 1 of Radiation Shielding Sheet

An urethane solution was applied on the surface of the embossed release paper at a thickness of 0.13 mm, and then heat-dried at a temperature of 105° C. for 30 sec in a heat drying chamber to form a base layer made of an urethane material.

The urethane solution applied on the embossed release paper for forming the base layer was a solution in which a solvent (DMF) was mixed with an urethane resin as described above, and the mixing ratio of the urethane resin and the solvent in the urethane solution for the base layer was 60 parts by weight of the solvent with respect to 100 parts by weight of the urethane resin. The urethane solution may be prepared by mixing a solvent (DMF) in an urethane resin having a viscosity of 50,000 to 80,000 cps. MEK and toluene may be used as the solvent. More specifically, at least one solvent selected from the group consisting of DMF, MEK, and toluene may be used.

In addition, a process of applying and then heat-drying (heat-drying for 50 sec at 110° C.) Example 1 (bismuth shielding material) of the aforementioned radiation shielding material on the base layer at a thickness disclosed in the following Table 1 for each step was continuously repeated five times to prepare Example 1 of a lead-free radiation shielding sheet having a single-layered base layer and a five-layered radiation shielding film (a first shielding film to a fifth shielding film) in the same as the structure illustrated in FIG. 1, and the thickness of the lead-free radiation shielding sheet prepared above was approximately 0.18 mm to 0.20 mm (average 0.19 mm).

At this time, the cumulative applying thickness (5 cumulative times) of the radiation shielding material for Example 1 was illustrated in the following Table 1 and a total thickness from the first step to the fifth step was 1.12 mm. When the applying thickness of the urethane solution for forming the base layer and the cumulative applying thickness of the radiation shielding material were summed, the total thickness was 1.25 mm, and the thickness was contracted by solvent evaporation to prepare a radiation shielding sheet having an average thickness of 0.19 mm as described above. It can be seen that pinholes were formed on the surface of the radiation shielding sheet according to Example 1 as the solvent was evaporated by heat-drying.

TABLE 1

| | Classification | | | | | |
|---|---|---|---|---|---|---|
| | Applying of | First | Second | Third | Fourth | Fifth |
| | urethane solution | applying | applying | applying | applying | applying |
| Applying | 0.13 mm | 0.17 mm | 0.2 mm | 0.2 mm | 0.30 mm | 0.35 mm |
| thickness | | | | | | |

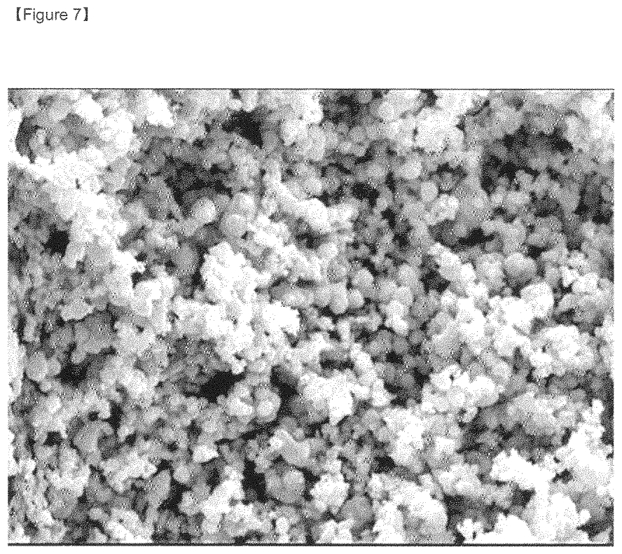

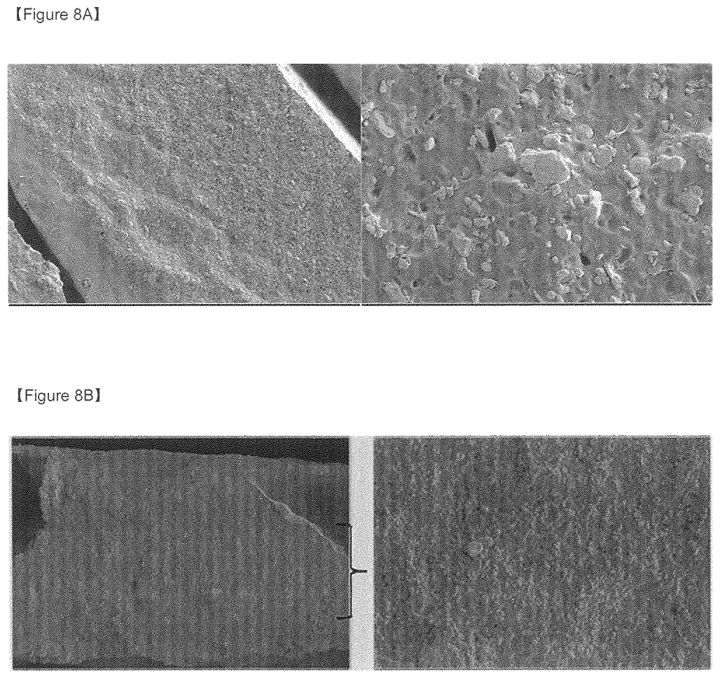

Then, the embossed release paper was peeled/removed from the base layer, and the radiation shielding performance of Example 1 of the lead-free radiation shielding sheet was examined, and a cross-sectional image of Example 1 (FIG. 8A) was obtained with a scanning electron microscope.

### Example 2 of Radiation Shielding Sheet

A base layer was formed on the surface of the embossed release paper using the same urethane solution as in Example 1 described above by the same applying thickness and heat drying method as in Example 1.

In addition, a process of applying/drying Example 2 (tungsten shielding material) of the above-described radiation shielding material on the base layer was sequentially repeated to prepare Example 2 of the radiation shielding sheet having a single-layered base layer and a five-layered radiation shielding film in the same manner as in Example 1 of the radiation shielding sheet (applying thickness and heat drying conditions are the same for each step).

At this time, the cumulative applying thickness of the radiation shielding material for Example 2 was illustrated in Table 1 and a total thickness from the first step to the fifth step was 1.12 mm. When the applying thickness of the urethane solution for forming the base layer and the cumulative applying thickness of the radiation shielding material were summed, the total thickness was 1.25 mm, and the thickness was contracted by solvent evaporation to prepare a radiation shielding sheet having an average thickness of 0.22 mm.

Then, the embossed release paper was peeled/removed from the base layer of Example 2, and the radiation shielding performance of Example 2 of the radiation shielding sheet was examined, and a cross-sectional image of Example 2 (FIG. 8B) was obtained with a scanning electron microscope. It can be seen that pinholes were formed on the surface of the radiation shielding sheet according to Example 2 as the solvent was evaporated by heat-drying.

### Example 3 of Radiation Shielding Sheet

A base layer was formed on the surface of the embossed release paper using the same urethane solution as in Example 1 described above by the same applying thickness and heat drying method as in Example 1.

In addition, the process of applying/drying Example 1 (bismuth shielding material) of the above-described radiation shielding material on the base layer was continuously performed twice to form a two-layered shielding film (B film) by the bismuth shielding material, and then the process of applying/drying Example 2 (tungsten shielding material) of the above-described radiation shielding material on the two-layered B film was continuously performed three times to form a three-layered shielding film (W film) by the tungsten shielding material.

The applying thickness and heat drying conditions of the radiation shielding material for each step (by order) were the same as those in Example 1, and Example 3 of the radiation shielding sheet having a single-layered base layer and a 5-layered radiation shielding film (2-layered B film/3-layered W film) was prepared.

That is, the cumulative applying thickness of the radiation shielding material for Example 3 was illustrated in Table 1 and a total thickness from the first step to the fifth step was 1.12 mm. When the applying thickness of the urethane solution for forming the base layer and the cumulative applying thickness of the radiation shielding material were summed, the total thickness was 1.25 mm, and the thickness was contracted by solvent evaporation to prepare a radiation shielding sheet having an average thickness of 0.225 mm.

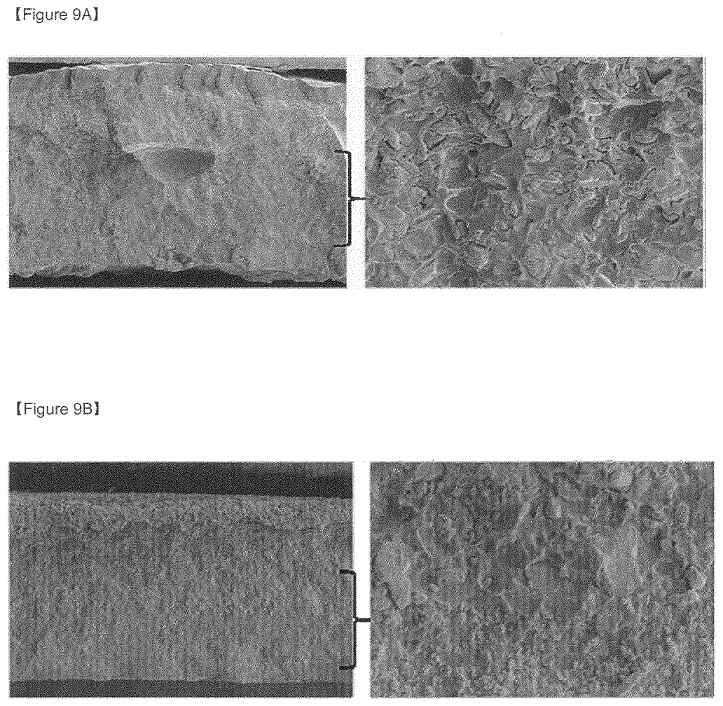

Then, the embossed release paper was peeled/removed from the base layer of Example 3, and the radiation shielding performance of Example 3 of the radiation shielding sheet was examined, and a cross-sectional image of Example 3 (FIG. 9A) was obtained with a scanning electron microscope. According to the scanning electron microscope, in Example 3, it can be seen that bismuth and tungsten are mixed at an interface where the B film and the W film meet. In addition, it can be seen through the electron microscope that pinholes are formed even on the surface of the radiation shielding sheet according to Example 3 as the solvent was evaporated by heat-drying.

### Example 4 of Radiation Shielding Sheet

A base layer was formed on the surface of the embossed release paper using the same urethane solution as in Example 1 described above by the same applying thickness and heat drying method as in Example 1.

In addition, the process of applying/drying Example 2 (tungsten shielding material) of the above-described radiation shielding material on the base layer was continuously performed twice to form a two-layered shielding film (W film) by the tungsten shielding material, and then the process of applying/drying Example 1 (bismuth shielding material) of the above-described radiation shielding material on the two-layered W film was continuously performed three times to form a three-layered shielding film (B film) by the bismuth shielding material.

The applying thickness and heat drying conditions of the radiation shielding material for each step (by order) were the same as those in Example 1, and Example 4 of the radiation shielding sheet having a single-layered base layer and a 5-layered radiation shielding film (2-layered W film/3-layered B film) was prepared.

That is, the cumulative applying thickness of the radiation shielding material for Example 4 was illustrated in Table 1 and a total thickness from the first step to the fifth step was 1.12 mm. When the applying thickness of the urethane solution for forming the base layer and the cumulative applying thickness of the tungsten shielding material and the bismuth shielding material were summed, the total thickness was 1.25 mm, and the thickness was contracted by solvent evaporation to prepare a radiation shielding sheet having an average thickness of 0.195 mm.

Then, the embossed release paper was peeled/removed from the base layer of Example 4, and the radiation shielding performance of Example 4 of the radiation shielding sheet was examined, and a cross-sectional image of Example 1[p1] (FIG. 9B) was obtained with a scanning electron microscope. It can be seen through the electron microscope that pinholes are formed even on the surface of the radiation shielding sheet according to Example 4 as the solvent was evaporated by heat-drying.

### Comparative Example of Radiation Shielding Sheet

A base layer was formed on the surface of the embossed release paper using the same urethane solution as in Example 1 described above by the same applying thickness and heat drying method as in Example 1.

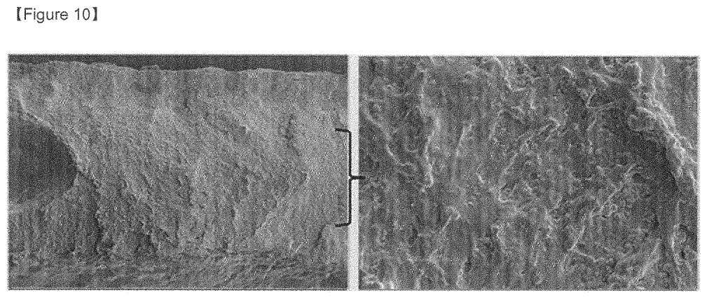

In addition, the process of applying at a thickness of 1.12 mm and drying Example 2 (tungsten shielding material) of the aforementioned radiation shielding material on the base layer once was performed to prepare Comparative Example of a radiation shielding sheet having a single-layered base layer and a single-layered radiation shielding film. For Comparative Example, the radiation shielding material applied in a single layer on the base layer was heat-dried for 250 sec at 110° C., and when the applying thickness of the urethane solution for forming the base layer and the applying thickness of the tungsten shielding material applied in the single layer were summed, the total thickness was 1.25 mm and the same as those in Examples above. The thickness was contracted to about ⅓ level by solvent evaporation to prepare a radiation shielding sheet having an average thickness of 0.39 mm and the thickness deviation for each part was greatly shown. In addition, a cross-sectional image (FIG. 10) of Comparative Example was obtained with a scanning electron microscope.

Therefore, like Comparative Example, since the shielding sheet manufactured by a single-layer applying method of applying the radiation shielding material once at the same thickness as the total cumulative applying thickness of a multi-layered thin film type is ununiform in thickness for each part, thick, and lack of flexibility, it can be seen that the compatibility is significantly reduced compared to Examples of the present invention in terms of physical properties.

### 3. Experimental Example of Radiation Shielding Sheet

The radiation shielding performance (shielding rate) for Examples 1 to 4 of the radiation shielding sheets was examined. An X-ray generator, an X-ray detector, and inspection conditions used for a shielding performance (shielding rate) test, that is, radiation exposure conditions are shown in [Table 2] below.

TABLE 2

| | |
|---|---|
| Radiation source | Heliodent Plus, Sinona Co, Bensheim, Germany |
| Detector | Multi-Detector XR(Magicmax Universal Multimeter) |
| | IBA, Schwarzenbruck, Germany |
| Exposure | Tube voltages of 60 and 70 kVp, Tube current of 7 |
| condition | mA |

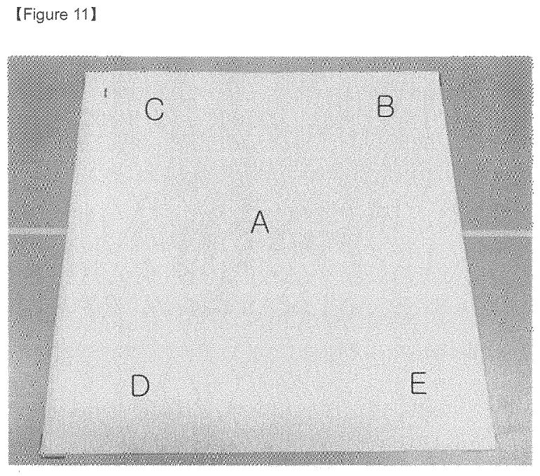

Examples 1 to 4 of the radiation shielding sheets were cut into squares of each having a size of 30 cm width and 30 cm length, respectively, as illustrated in FIG. 11 to obtain test specimens. In the shielding rate test, a distance between the radiation source and the detector was 30 cm, and an exposure time was 0.2 sec, and equipment shown in [Table 2] below was used. The dose was measured in a total of 5 areas (a central area and four edge areas; points A, B, C, D, and E of FIG. 11) for one specimen and the radiation shielding rates and standard deviations were derived. The dose measurement was repeated a total of 6 times, and the radiation shielding rate was calculated by [Equation 1] below.

S=(I₂−I₁)I₁×100 [Equation 1]

(S represents a shielding rate (%), I₁ represents a measured dose without shielding sheet, and I₂ represents a measured dose through shielding sheet (test specimen).

Radiation shielding rates and standard deviations of Examples 1 to 4 and Comparative Example of the radiation shielding sheets measured by the above-mentioned equipment and test conditions are as shown in [Table 3] below, and in an order of test results (Example 4>Example 3>Example 2>Example 1), the shielding performance of Example 4 was highest.

TABLE 3

| Classification | | Example 1 | Example 2 | Example 3 | Example 4 |
|---|---|---|---|---|---|
| Shielding rate | 60 kVp | 68.1 | 72.6 | 75.5 | 83.8 |
| (%) | 70 kVp | 63.3 | 67.0 | 70.7 | 79.0 |

| | | | | |
|---|---|---|---|---|
| Standard deviation | 0.016 | 0.006 | 0.005 | 0.006 |
| Lead equivalent (mmPb) | 0.046 | 0.050 | 0.056 | 0.079 |

In the test specimens according to Examples 1 to 4, it can be seen that the standard deviation is small and the radiation shielding powder is evenly dispersed. Even in a scanning electron microscope (FIGS. 8 to 10), Examples 1 to 4 showed that the radiation shielding powder was relatively even, whereas in the case of Comparative Example, it can be seen that the radiation shielding powder is not evenly dispersed. In addition, even in a physical property test, Examples showed excellent performance, and in particular, excellent performance of 10,000 cycles in an abrasion resistance test (ISO 12947-2 test method) and 1,000 cycles or more in a flexibility (stiffness) test (ISO 5402-1 test method) can be confirmed.

As described above, the prepared embodiments of the present invention have been described as above and a fact that the present invention can be materialized in other specific forms without departing from the gist or scope of the present invention in addition to the above described embodiments will be apparent to those skilled in the art.

Therefore, the aforementioned embodiments are not limited but should be considered to be illustrative, and as a result, the present invention is not limited to the above description and may be modified within the scope of the appended claims and a range equivalent thereto.

### INDUSTRIAL APPLICABILITY

The present invention relates to a radiation protective material for shielding a radiation, and can be used as a radiation shielding material in various fields, such as radiation related fields, for example, medical protective clothing, industrial protective materials for nuclear power plants, protective clothing, household protective clothing, other test devices using radiation, etc.

1. A method for manufacturing a lead-free radiation shielding sheet comprising:
a film laminating step of forming a multi-layered radiation shielding film on one side of a base material by repeating a process of sequentially applying to laminate, drying, and integrating a radiation shielding material containing a radiation shielding powder and a binder for forming a film to be mixed with each other on one side of the base material for forming a radiation shielding sheet.

2. The method for manufacturing the lead-free radiation shielding sheet of claim 1, wherein the film laminating step comprises a shielding material applying step of sequentially applying and drying the radiation shielding material on one side of the base material a plurality of times so that the radiation shielding film has a multi-layered structure of at least three layers.

3. The method for manufacturing the lead-free radiation shielding sheet of claim 2, wherein the shielding material applying step comprises forming an N-layered radiation shielding film by sequentially applying and drying the radiation shielding material on one side of the base material N (3≤N≤10) times.

4. The method for manufacturing the lead-free radiation shielding sheet of claim 1, wherein the film laminating step comprises a shielding material applying step of repeating a process of sequentially applying at a thickness of 0.05 mm to 0.50 mm and drying the radiation shielding material on one side of the base material a plurality of times.

5. The method for manufacturing the lead-free radiation shielding sheet of claim 4, wherein the shielding material applying step comprises an inner film forming step of performing a process of sequentially applying at a thickness of 0.1 mm to 0.30 mm and drying the radiation shielding material on one side of the base material at least twice.

6. The method for manufacturing the lead-free radiation shielding sheet of claim 4, wherein the shielding material applying step comprises
a first film forming step of forming a first shielding film by applying at a thickness of 0.1 mm to 0.3 mm and drying the radiation shielding material on one side of the base material;
a second film forming step of forming a second shielding film by applying at a thickness of 0.1 mm to 0.3 mm and then drying the radiation shielding material on the first shielding film;
a third film forming step of forming a third shielding film by applying at a thickness of 0.1 mm to 0.3 mm and then drying the radiation shielding material on the second shielding film;
a fourth film forming step of forming a fourth shielding film by applying at a thickness of 0.1 mm to 0.4 mm and then drying the radiation shielding material on the third shielding film; and
a fifth film forming step of forming a fifth shielding film by applying at a thickness of 0.2 mm to 0.45 mm and then drying the radiation shielding solution on the fourth shielding film.

7. The method for manufacturing the lead-free radiation shielding sheet of claim 4, wherein the shielding material applying step is to apply and laminate sequentially the radiation shielding material on one side of the base material N (4≤N≤8) times so that a total cumulative applying thickness of the radiation shielding material is 0.5 mm to 2.0 mm.

8. The method for manufacturing the lead-free radiation shielding sheet of claim 4, wherein the shielding material applying step comprises a front film forming step comprising a first applying step of forming a first radiation shielding film by initially applying the radiation shielding material on one side of the base material and a rear film forming step of forming a rear radiation shielding film of at least one layer by additionally applying the radiation shielding material on a front radiation shielding film formed by the front film forming step, wherein the rear film forming step comprises at least one applying step of the radiation shielding material at a different applying thickness as compared with the first applying step.

9. The method for manufacturing the lead-free radiation shielding sheet of claim 8, wherein in an individual applying step of the rear film forming step, the radiation shielding material is applied thicker than that of the first applying step.

10. The method for manufacturing the lead-free radiation shielding sheet of claim 8, wherein in the rear film forming step, the process of applying and drying the radiation shielding material is sequentially performed a plurality of times; and in the last applying step of forming a surface layer of the radiation shielding film in the rear film forming step, the applying thickness of the radiation shielding material is thickest.

11. The method for manufacturing the lead-free radiation shielding sheet of claim 1, wherein the radiation shielding powder comprises at least one selected from the group consisting of tungsten, bismuth, barium sulfate, antimony, boron, or a compound containing the same.

12. The method for manufacturing the lead-free radiation shielding sheet of claim 1, wherein the binder comprises at least one selected from the group consisting of an urethane resin, an acrylic resin, an epoxy resin, or a polyester resin.

13. The method for manufacturing the lead-free radiation shielding sheet of claim 1, wherein the radiation shielding material contains at least one powder of tungsten and a tungsten compound as the radiation shielding powder; and
the film laminating step comprises a shielding material applying step of forming the multi-layered radiation shielding film by sequentially applying the radiation shielding material containing at least one powder of tungsten and a tungsten compound on one side of the base material.

14. The method for manufacturing the lead-free radiation shielding sheet of claim 1, wherein the radiation shielding material comprises a tungsten shielding material containing at least one powder of tungsten and a tungsten compound as the radiation shielding powder and a bismuth shielding material containing at least one shielding powder of bismuth and a bismuth compound as the radiation shielding powder; and
the film laminating step comprises a tungsten film forming step of forming at least one layer of the radiation shielding film with the tungsten shielding material, and
a bismuth film forming step of forming at least one layer of the radiation shielding film with the bismuth shielding material, before or after the tungsten film forming step.

15. The method for manufacturing the lead-free radiation shielding sheet of claim 14, wherein the tungsten film forming step comprises a step of forming at least two layers of the radiation shielding film with the tungsten shielding material in a surface contact state; and
the bismuth film forming step is performed before or after the tungsten shielding material applying step and comprises a step of forming at least two layers of the radiation shielding film with the bismuth shielding material in a surface contact state.

16. The method for manufacturing the lead-free radiation shielding sheet of claim 1, further comprising:
before the film laminating step,
a base coating step of forming a base layer for enhancing adhesion of the radiation shielding film on one surface of the base material applied with the radiation shielding material.

17. The method for manufacturing the lead-free radiation shielding sheet of claim 16, wherein the base coating step comprises a step of directly applying the liquid material for forming the base layer on one surface of the base material at a thickness of 0.05 mm to 0.2 mm.
